(19) United States
(12) Patent Application Publication
(10) Pub. No.: US 2006/0140882 A1
(43) Pub. Date: Jun. 29, 2006

(54) Two phase whitening oral care composition

(76) Inventors: Gary E. Tambs, Belle Mead, NJ (US); Evangelia S. Arvanitidou, Princeton, NJ (US); Nuran Nabi, Cranbury, NJ (US)

Correspondence Address: COLGATE-PALMOLIVE COMPANY, 909 RIVER ROAD, PISCATAWAY, NJ, 08855, US

(21) Appl. No.: 11/025,582

(22) Filed: Dec. 29, 2004

(51) Int. Cl. A61K 8/22

(52) U.S. Cl. 424/53

(57) ABSTRACT

An oral care (toothpaste) amalgam composition has first and second aqueous phases. The first phase has a measured pH of from about 7 to about 9 and contains 2,4,4′-trichloro-2′-hydroxydiphenyl ether and a methylvinyl ether-maleic anhydride copolymer antibacterial-enhancing agent for the ether. The second aqueous phase has a measured pH of from about 3.5 to about 6.5 and contains hydrogen peroxide and an antioxidant for the hydrogen peroxide. Sodium fluoride, silica, and sodium lauryl sulfate are optionally independently present in at least one of the first and second phases.

The present invention relates to oral care compositions and methods. In particular, the present invention includes compositions and methods for cleaning of teeth.

Many individuals desire a “bright” smile and white teeth, and consider dull and stained teeth cosmetically unattractive. Unfortunately, without preventive or remedial measures, stained teeth are almost inevitable due to the absorbent nature of dental material. Everyday activities such as smoking or other oral use of tobacco products, and eating, chewing or drinking certain foods and beverages (in particular coffee, tea and red wine), cause undesirable staining of surfaces of teeth. Staining can also result from microbial activity, including that associated with dental plaque. The chromogens or color causing substances in these materials become part of the pellicle layer and can permeate the enamel layer. Even with regular brushing and flossing, years of chromogen accumulation can impart noticeable tooth discoloration.

A tooth is comprised of an inner dentin layer and an outer hard enamel layer that is the protective layer of the tooth. The enamel layer of a tooth is naturally opaque, and white or a slightly off-white color. The enamel layer is composed of hydroxyapatite mineral crystals that create a somewhat porous surface. These hydroxyapatite crystals form microscopic hexagonal rods or prisms that make up the enamel surface. As a result, the surface of the enamel presents microscopic spaces or pores between the prisms. Without limiting the mechanism, function or utility of present invention, it is believed that this porous nature of the enamel is where discoloring substances permeate the enamel and discolor the teeth.

There are a variety of compositions described in the art for preventing or treating the discoloration and/or the damage of teeth. In particular, to combat staining and brighten or restore the natural enamel color, a variety of products containing bleaching materials are commercially available for professional and consumer use. The most commonly accepted chemicals used in teeth whitening today are peroxides. Peroxides are generally deemed safe from a physiological standpoint, and can be effective to whiten teeth. Such peroxides include hydrogen peroxide, carbamide peroxide, sodium perborate, and sodium percarbonate. When these peroxides are in appropriate contact with teeth they will usually oxidize stains, rendering the teeth whiter.

Current home treatment methods include abrasive toothpastes, toothpastes that produce oxides, whitening gels for use with a dental tray and whitening strips. The effectiveness of such techniques depends on a variety of factors including the type and intensity of the stain, the type of bleaching agent, contact time of the bleaching agent on the teeth, the amount of available bleaching active in the composition, the ability of the bleaching agent to penetrate the tooth enamel, interactive effects of various components in clinical efficacy of toothpaste formulations respective to both the appearance of teeth and the general health of teeth, and consumer compliance. Effectiveness is also dependant on the amount of bleaching active in the composition, the ability of the active to be released during use, and the stability of the active in the product. However, the effectiveness of many of these treatments is adversely affected because of deficiencies in one or more factors relating to the composition and consumer compliance.

### SUMMARY

The present invention provides oral care compositions. Embodiments include oral care compositions, comprising:
(a) a first phase comprising a halogenated diphenyl ether (preferably 2,4,4′-trichloro-2′-hydroxydiphenyl ether), an antibacterial enhancing agent (preferably a methylvinyl ether-maleic anhydride copolymer), and a first orally acceptable aqueous vehicle; and
(b) a second phase comprising a peroxide whitening agent (preferably hydrogen peroxide), an antioxidant, and a second orally acceptable aqueous vehicle; where
(c) the first phase has a measured pH of from about 7 to about 9, the second phase has a measured pH of from about 3.5 to about 6.5, the first phase provides at least one first portion of an amalgam, the second phase provides at least one second portion of the amalgam, and at least one first portion is in fluid interface with at least on second portion in the amalgam.

In one aspect, the methylvinyl ether-maleic anhydride copolymer antibacterial-enhancing agent has a molecular weight of about 100 to about 1,000,000.

In another aspect, the antioxidant is butylated hydroxy toluene. In another aspect sodium fluoride, silica, and sodium lauryl sulfate are independently present in at least one of the first phase and second phases.

The invention is also for a method for making an oral care composition, comprising:
(a) admixing a first dentifrice comprising clinically efficacious 2,4,4′-trichloro-2′-hydroxydiphenyl ether and a methylvinyl ether-maleic anhydride copolymer antibacterial-enhancing agent for the ether in a first orally acceptable aqueous vehicle;
(b) admixing a second dentifrice comprising clinically efficacious amount of hydrogen peroxide and an antioxidant for the hydrogen peroxide in a second orally acceptable aqueous vehicle; and
(c) blending an amalgam of the first and the second dentifrices; where
(d) the antibacterial-enhancing agent is admixed in the first dentifrice in an amount having a weight ratio of from about 0.15 to about 32 respective to the amount of the ether, the first dentifrice is admixed to have a measured pH of from about 7 to about 9, the second dentifrice is admixed to have a measured pH of from about 3.5 to about 6.5, the first dentifrice provides at least one first portion of the amalgam, the second dentifrice provides at least one second portion of the amalgam, and at least one first portion is blended to be in fluid interface with at least one second portion in the amalgam.

In one aspect, the invention includes the further steps of storing the first dentifrice in a first enclosure and of storing the second dentifrice in a second enclosure. The blending step then further comprises expelling the first dentifrice from the first enclosure and expelling the second dentifrice from the second enclosure so that the first dentifrice and the second dentifrice are expelled to provide the amalgam.

In another aspect, an oral care kit is provided, comprising:
a) a first chamber having a first outlet in fluid communication with the first chamber for discharge of a first dentifrice from the first chamber; and
(b) a second chamber having a second outlet in fluid communication with the second chamber for discharge of a second dentifrice from the second chamber;
(c) where the first dentifrice comprises clinically efficacious 2,4,4′-trichloro-2′-hydroxydiphenyl ether and a methylvinyl ether-maleic anhydride copolymer antibacterial-enhancing agent for the ether admixed in a first orally acceptable aqueous vehicle, the second dentifrice comprises clinically efficacious hydrogen peroxide and an antioxidant for the hydrogen peroxide admixed in a second orally acceptable aqueous vehicle, the antibacterial-enhancing agent is in the first dentifrice in an amount having a weight ratio of from about 0.15 to about 32 respective to the amount of the ether, the first dentifrice has a measured pH of from about 7 to about 9, and the second dentifrice has a measured pH of from about 3.5 to about 6.5, and the second outlet is proximate to the first outlet so that, during simultaneous discharge of the first dentifrice from the first chamber through the first outlet and of the second dentifrice from the second chamber through the second outlet, discharged first dentifrice fluidly interfaces with discharged second dentifrice to yield an amalgam (such as a toothpaste) such that the discharged first dentifrice provides at least one first portion of the amalgam, the discharged second dentifrice provides at least one second portion of the amalgam, and at least one first portion is in fluid interface with at least one second portion. In one aspect, the simultaneous discharge of the first dentifrice (from the first chamber through the first outlet) and of the second dentifrice (from the second chamber through the second outlet) provides essentially equivalent volumetric flows of the discharged first dentifrice and the discharged second dentifrice.

In yet another aspect, a method for cleaning teeth of a human or other animal subject is provided by brushing the teeth of the human or animal subject with about 2 grams of toothpaste according to the above formulations.

It has been discovered that compositions and methods of this invention afford advantages over oral care compositions among known in the art. Further uses, benefits and embodiments of the present invention are apparent from the description set forth herein.

### DESCRIPTION

The following definitions and non-limiting guidelines must be considered in reviewing the description of this invention set forth herein. The headings (such as “Introduction” and “Summary,”) and sub-headings (such as “Compositions” and “Methods”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

As used herein, the term “about,” when applied to the value for a parameter of a composition or method of this invention, indicates that the calculation or the measurement of the value allows some slight imprecision without having a substantial effect on the chemical or physical attributes of the composition or method. If, for some reason, the imprecision provided by “about” is not otherwise understood in the art with this ordinary meaning, then “about” as used herein indicates a possible variation of up to 5% in the value.

As referred to herein, all compositional percentages are by weight of the total composition, unless otherwise specified.

Compositions

The present invention provides oral care compositions and methods for administration or application to, or use with, a human or other animal subject. As referred to herein, an “oral care composition” is any composition that is suitable for administration or application to the oral cavity a human or animal subject for enhancing the health, hygiene or appearance of the subject, preferably providing such benefits as: the prevention or treatment of a condition or disorder of the teeth, gums, mucosa or other hard or soft tissue of the oral cavity; the prevention or treatment of a systemic condition or disorder; the provision of sensory, decorative or cosmetic benefits; and combinations thereof. In various preferred embodiments, an oral care composition is not intentionally swallowed, but is rather retained in the oral cavity for a time sufficient to effect the intended utility. Preferably, specific materials and compositions to be used in this invention are, accordingly, pharmaceutically- or cosmetically-acceptable. As used herein, such a “pharmaceutically acceptable”, “clinically efficacious, or “cosmetically acceptable” component or “orally acceptable vehicle” is one that is suitable for use with humans and/or animals to provide the desired therapeutic, prophylactic, sensory, decorative, or cosmetic benefit without undue adverse side effects (such as toxicity, irritation, and allergic response) commensurate with a reasonable benefit/risk ratio.

In particular, the present invention provides oral care compositions, comprising:
(a) a first phase comprising a halogenated diphenyl ether (preferably 2,4,4′-trichloro-2′-hydroxydiphenyl ether), an antibacterial enhancing agent (preferably a methylvinyl ether-maleic anhydride copolymer), and a first orally acceptable aqueous vehicle; and
(b) a second phase comprising a peroxide whitening agent (preferably hydrogen peroxide), an antioxidant, and a second orally acceptable aqueous vehicle; where
(c) the first phase has a measured pH of from about 7 to about 9, the second phase has a measured pH of from about 3.5 to about 6.5, the first phase provides at least one first portion of an amalgam, the second phase provides at least one second portion of the amalgam, and at least one first portion is in fluid interface with at least on second portion in the amalgam.

Oral care compositional and method embodiments described herein particularly reflect the beneficial efficacy of a surprising find that toothpaste formulations essentially devoid of polyethylene glycol and providing a clinically efficacious halogenated diphenyl ether and as an antibacterial active and hydrogen peroxide as a whitening agent show enhanced antibacterial efficacy when compared to toothpaste formulations containing polyethylene glycol, halogenated diphenyl ether, and hydrogen peroxide. In this regard, polyethylene glycols (PEGs) have traditionally provided efficacy in toothpaste formulations in foam modulation (control of amount, thickness and/or stability of foam generated by the composition upon agitation) in moisture control (as a humectant), astringency mask, and facilitating agent for enhanced flavor perception.

Halogenated Diphenyl Ether:

The first phase of the composition of the invention comprises a phenolic antimicrobial, preferably a halogenated diphenylether. Preferred diphenylethers useful herein include triclosan (2,4,4′-trichloro-2′-hydroxydiphenyl ether), triclosan monophosphate or 2,2′-dihydroxy-5,5′-dibromodiphenylether. Triclosan is a particularly preferred diphenylether.

Antibacterial-Enhancing Agents:

The enhancing agents (EA) of the present invention can include those that are characterized as having utility as denture adhesives or fixatives or dental cements. The enhancing agent include polymers or copolymers, which terms are entirely generic, thus including for example oligomers, homopolymers, copolymers of two or more monomers, ionomers, block copolymers, graft copolymers, cross-linked polymers and copolymers, and the like. The EA may be natural or synthetic, and water (saliva) soluble or swellable (hydratable, hydrogel forming) polymer or copolymer. The EA can be selected to have various sizes, such as an (weight) average molecular weight (MW) of: from about 100 to about 1,000,000; from about 1,000 to about 1,000,000; or from about 2,000-2,500 to about 250,000-500,000.

The delivery enhancing groups of the EA can be any of those listed in U.S. Pat. Nos. 5,538,715 and 5,776,435, which are incorporated by reference. In various embodiments, the delivery-enhancing groups are preferably acidic such as sulfonic, phosphinic, or more preferably phosphonic or carboxylic, or a salt thereof, e.g. alkali metal or ammonium. The retention enhancing group(s) can be any organic retention-enhancing group, for example, those that have the formula —(X)n-R wherein X is O, N, S, SO, SO₂, P, PO or Si or the like, R is hydrophobic alkyl, alkenyl, acyl, aryl, alkaryl, aralkyl, heterocyclic or their inert-substituted derivatives, and n is zero or one or more. The aforesaid “inert-substituted derivatives”, are intended to include substituents on R which are generally non-hydrophilic and do not significantly interfere with the desired functions of the EA as enhancing the delivery of the mixture (anti-bacterial agent) to, and retention thereof on, oral surfaces such as halo, e.g., Cl, Br, I, and carbo and the like.

The EA can be a synthetic anionic polymeric or linear anionic polymeric polycarboxylate having an average MW of from about 100 to about 1,000,000, or from about 1,000 to about 1,000,000, and can be present in the oral composition from about 0.0005 to about 5% by weight, from about 0.005 to about 4% by weight, or from about 0.05 to about 3% by weight. The EA can be an anionic copolymer of maleic acid or anhydride with another ethylenically unsaturated polymerizable monomer. The EA can be a copolymer of maleic acid or anhydride with methyl vinyl ether, such as any one or more of the forms of GANTREZ (available from ISP of Wayne, N.J.).

The delivery enhancing groups of the EA can also be various phosphonates. Such phosphonate-type EA's can have an average MW from about 100 to about 1,000,000 or from about 1,000 to about 1,000,000. The EA can be a polyvinyl phosphonate and/or alkali metal polyvinyl phosphonate and/or ammonium polyvinyl phosphonate of MW about 1000 or more. The phosphonate-type EA can be present in the oral composition from about 0.0005 to about 4% by weight. The EA can be a poly(β-styrenephosphonate), poly(α-styrenephosphonate), copoly(α,β-styrenephosphonate) or another copolymer of α-or β-styrenephosphonate with another polymerizable ethylenically unsaturated monomer, such as copoly (β-styrenephosphonate/vinylphosphonate). The phosphonate-type EA can have an average MW of from about 2,000 to about 30,000.

The antibacterial-enhancing agent is in the first phase (the phase having the ether) in an amount having a weight ratio of from about 0.1:1 to about 35:1, respective to the ether, optionally from about 0.15:1 to about 32:1, optionally from about 1:1 to about 10:1, optionally from about 2:1 to about 8:1, optionally from about 5:1 to about 7:1. In various embodiments, the antibacterial-enhancing agent is in the first phase in an amount having a weight ratio of from about 0.15 to about 32 respective to the halogenated diphenyl ether.

In one embodiment, the methylvinyl ether-maleic anhydride copolymer antibacterial-enhancing agent has a molecular weight of about 100 to about 1,000,000. In one embodiment, the methylvinyl ether-maleic anhydride copolymer antibacterial agent has the structural formula 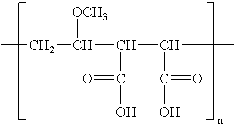
where n provides molecular weight in the agent from about 60,000 to about 500,000. In another embodiment, the silica oral polishing agent is high cleaning silica.

In one embodiment, the antioxidant is butylated hydroxy toluene. In one embodiment, sodium fluoride is in the first phase and/or the second phase. In one embodiment, silica is in the first phase and/or the second phase. In one embodiment, sodium lauryl sulfate is in the first phase and/or the second phase.

Peroxide Compound:

The present invention provides compositions comprising a peroxide compound. A “peroxide compound” is any oxidizing compound comprising a bivalent oxygen-oxygen group. Peroxide components include peroxides and hydroperoxides, such as hydrogen peroxide (the preferred peroxide component in the embodiments), peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as percarbonate and perborate salts of alkali and alkaline earth metals such as lithium, potassium, sodium, magnesium, calcium and barium, and mixtures thereof. In one embodiment, the peroxide compound comprises hydrogen peroxide. In one embodiment, the peroxide component consists essentially of hydrogen peroxide. The peroxide compound is preferably present at a level of from about 0.5 to about 4 weight percent in one phase of the composition.

Orally Acceptable Carrier

The present invention provides compositions comprising an orally acceptable carrier. As used herein, an “orally acceptable carrier” refers to a material or combination of materials that are safe for use in the compositions of the present invention, commensurate with a reasonable benefit/risk ratio, with which halogenated diphenyl ether, antibacterial enhancing agent, peroxide compound, and an antioxidant for the hydrogen peroxide may be associated while retaining significant clinical efficacy. Preferably, the carrier does not substantially reduce the efficacy of the halogenated diphenyl ether or peroxide compound. In various embodiments, the carrier is operable to sufficiently adhere the dentifrice against surfaces within the oral cavity to which the composition is administered, without concomitant use of a dental tray, mouthpiece, tape, or similar appliance. In various preferred embodiments, an oral care composition is not intentionally swallowed, but is rather retained in the oral cavity for a time sufficient to effect the intended utility. Preferably, specific materials and compositions to be used in this invention are, accordingly, pharmaceutically- or cosmetically-acceptable. As used herein, such a “pharmaceutically acceptable” or “cosmetically acceptable” component is one that is suitable for use with humans and/or animals to provide the desired therapeutic, prophylactic, sensory, decorative, or cosmetic benefit without undue adverse side effects (such as toxicity, irritation, and allergic response) commensurate with a reasonable benefit/risk ratio.

The first of the two admixtures provides a first phase for at least one first portion of the “amalgam” composition, the second of the two admixtures provides a second phase for at least one second portion of the amalgam (at least one first portion is in fluid interface with at least one second portion in the amalgam). In all embodiments, the hydrogen peroxide and the halogenated diphenyl ether are first formulated to be physically separated in two separate aqueous admixtures (for maximum stability of the peroxide), each of the two aqueous admixtures is stored in a physically separate chamber (such as provided in a dual-compartment tube of toothpaste), and the two aqueous admixtures are blended together into an amalgam when the anticipated time between amalgam formation and application of the amalgam to the teeth is sufficiently minimal such that detrimental decomposition of the hydrogen peroxide and/or the halogenated diphenyl ether do not occur prior to use. In this regard, the first dentifrice (with the halogenated diphenyl ether) provides at least one first portion (or compositional phase) of the amalgam and the second dentifrice (having the hydrogen peroxide) provides at least one second portion (or compositional phase) of the amalgam. It is preferred that the first phase be at a pH of from about 7 to about 9, insofar as an acidic pH is detrimental to the halogenated diphenyl ether; and it is preferred that the second phase be at a pH of from about 3.5 to about 6.5, insofar as a basic pH is detrimental to the hydrogen peroxide. When the first dentifrice is expelled from the first enclosure of the dual-compartment tube to provide a first portion of the amalgam (on a toothbrush) and the second dentifrice is expelled from the second enclosure of the dual-compartment tube to provide a second portion of the amalgam, the chemical properties (such as pH) within each portion of the amalgam continue to be generally independently sustained as were previously intrinsic to the respective first and second enclosures even though the two portions (two phases) are in fluid interface. The general intermixing of the phases into a common chemical property environment occurs on the tooth surface only as the two phases of the amalgam are intermixed by a brushing action. Indeed, some deposition of hydrogen peroxide and halogenated diphenyl ether molecules from the independent phases of the amalgam will occur on the tooth surface just prior to intermixing of the portions commensurate with actual brushing. In this manner, the properties of the actives are essentially sustained up to the time of application to the tooth surface.

More specifically, one formulation embodiment provides an oral care composition having a first phase of clinically efficacious halogenated diphenyl ether and a antibacterial-enhancing agent for the ether admixed in a first orally acceptable aqueous vehicle and a second phase comprising clinically efficacious hydrogen peroxide and an antioxidant for the hydrogen peroxide admixed in a second orally acceptable aqueous vehicle. The first phase has a measured pH of from about 7 to about 9, and the second phase has a measured pH of from about 3.5 to about 6.5.

In one embodiment, the first and second phases are independently formulated such that the halogenated diphenyl ether is admixed into the first orally acceptable aqueous vehicle and such that the hydrogen peroxide is admixed into the second orally acceptable aqueous vehicle. The first and second phases are then kept from physical chemical interaction prior to a brief period prior to application to a tooth surface. At that time, the first and second phases are combined together (usually on a toothbrush) as an amalgam (a compound of different things) where the first phase provides at least one first portion of the amalgam, the second phase provides at least one second portion of the amalgam, and at least one of the first portions is in fluid interface with at least one of the second portions in the amalgam. Usually, this is achieved by use of a dual-compartment toothpaste tube (oral care kit) where two approximately equivalent volumetric portions from each of the two compartments are simultaneously fluidly expelled from the compartments and extruded in fluid interface onto a toothbrush under the presumption that the resulting amalgam dentifrice is to be immediately applied to the teeth by the user.

Materials among those that are useful in carriers include adhesion agents, viscosity modifiers, diluents, surfactants, foam modulators, peroxide activators, peroxide stability agents, abrasives, pH modifying agents, humectants, mouth feel agents, sweeteners, flavorants, colorants, and combinations thereof. It is understood that while general attributes of each of the above categories of materials may differ, there may be some common attributes and any given material may serve multiple purposes within two or more of such categories of materials. Preferably, such carrier materials are selected for compatibility with the peroxide components and with other ingredients of the composition.

In various embodiments, the carrier comprises an adhesion agent. As referred to herein, an adhesion agent is a material or combination of materials that enhance the retention of the peroxide component on the oral cavity surface onto which the composition is applied. Such adhesion agents include adhesives, film forming materials, viscosity enhancers and combinations thereof. Such materials include hydrophilic organic polymers, hydrophobic organic polymers, silicone gums, silicone adhesives, silicas, and combinations thereof.

Thickening agents among those useful herein include carboxyvinyl polymers, carrageenans (also known as Irish moss and more particularly iota-carrageenan), cellulosic polymers such as hydroxyethylcellulose, carboxymethylcellulose (carmellose) and salts thereof (e.g., carmellose sodium), natural gums such as karaya, xanthan, gum arabic and tragacanth, colloidal magnesium aluminum silicate, colloidal silica, and mixtures thereof. As previously noted, the embodiments are essentially devoid of polyethylene glycol.

Viscosity modifiers among those useful herein include mineral oil, petrolatum, clays and organomodified clays, silica, and mixtures thereof. In various embodiments, such viscosity modifiers are operable to inhibit settling or separation of ingredients or to promote redispersibility upon agitation of a liquid composition. As previously noted, the embodiments are essentially devoid of polyethylene glycol.

Diluents among those useful herein include materials or combinations of materials that are operable to solubilize and/or suspend other components of the composition. In various embodiments, diluents are operable to adjust the viscosity of the composition, optionally in conjunction with viscosity modifiers (as discussed herein) and other components of the composition.

Surfactants among those useful herein include anionic, nonionic, and amphoteric surfactants. Surfactants may be used, for example, to provide enhanced active delivery of the formulation, to help in cleaning the oral cavity surfaces through detergency, and to provide foam upon agitation, e.g., during brushing with a dentifrice composition of the invention. Suitable anionic surfactants include water-soluble salts of C₈₋₂₀ alkyl sulfates, sulfonated monoglycerides of C₈₋₂₀ fatty acids, sarcosinates, taurates, and mixtures thereof. Illustrative examples of these and other surfactants are sodium lauryl sulfate, sodium coconut monoglyceride sulfonate, sodium lauryl sarcosinate, sodium lauryl isoethionate, sodium laureth carboxylate and sodium dodecyl benzenesulfonate, and mixtures thereof. Suitable nonionic surfactants include poloxamers, polyoxyethylene sorbitan esters, fatty alcohol ethoxylates, alkylphenol ethoxylates, tertiary amine oxides, tertiary phosphine oxides, dialkyl sulfoxides, and mixtures thereof. As previously noted, the embodiments are essentially devoid of polyethylene glycol.

Humectants useful herein include polyhydric alcohols such as glycerin, sorbitol, or xylitol. In various embodiments, humectants are operable to prevent hardening of paste or gel compositions upon exposure to air. In various embodiments humectants also function as sweeteners. As previously noted, the embodiments are essentially devoid of polyethylene glycol.

Peroxide activators such as sodium bicarbonate, sodium carbonate, and/or manganese gluconate may be incorporated in the compositions of the present invention. The activator is relatively nonactive with the peroxide whitening agent in nonaqueous liquid compositions. In various embodiments, the activator is operable to react with the peroxide to release oxygen when the liquid whitening composition applied to the teeth is contacted with saliva in the oral cavity. The peroxide activator is optionally present in embodiments of this invention at a concentration of about 0.001% to about 50% (preferably 0.1% to about 50%).

Agents for modifying pH (pH modifying agents) among those useful herein include acidifying agents to lower pH, basifying agents to raise pH, and buffering agents to control pH within a desired range. For example, one or more compounds selected from acidifying, basifying and buffering agents can be included to provide a pH of about 3.5 to about 9, from about 3.5 to about 6.5, or from about 7 to about 9. Any orally acceptable pH modifying agent can be used, including carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole, and mixtures thereof. One or more pH modifying agents are optionally present in a total amount effective to maintain the composition or individual phases and/or portions of the composition in an orally acceptable pH range. With respect to pH, it is to be noted that the pH is a measured pH, or that pH indicated on a pH measuring instrument when the measuring sensor is placed within the material for which a pH measurement is desired.

Mouth-feel agents include materials that impart a desirable texture or other feeling during use of the composition. Such agents include bicarbonate salts, which in various embodiments impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate, and mixtures thereof.

Flavorants among those useful herein include any material or mixture of materials operable to enhance the taste of the composition. Any orally acceptable natural or synthetic flavorant can be used, such as flavoring oils, flavoring aldehydes, esters, alcohols, similar materials, and combinations thereof. Flavorants include vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavorants herein are ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), and mixtures thereof.

Sweeteners among those useful herein include orally acceptable natural or artificial, nutritive or non-nutritive sweeteners. Such sweeteners include dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones, and mixtures thereof. One or more sweeteners are optionally present in a total amount depending strongly on the particular sweetener(s) selected, but typically at levels of from about 0.001% to about 5%, optionally from about 0.01% to about 1%.

Colorants among those useful herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5%.

Optional Active Materials:

The compositions of the present invention optionally comprise additional active materials, which are operable for the prevention or treatment of a condition or disorder of hard or soft tissue of the oral cavity, the prevention or treatment of a physiological disorder or condition, or to provide a cosmetic benefit. In various embodiments, the active is a “systemic active” which is operable to treat or prevent a disorder that, in whole or in part, is not a disorder of the oral cavity. In various embodiments, the active is an “oral care active” operable to treat or prevent a disorder or provide a cosmetic benefit within the oral cavity (e.g., to the teeth, gingiva or other hard or soft tissue of the oral cavity). Oral care actives among those useful herein include whitening agents, anticaries agents, tartar control agents, antiplaque agents, antigingivitis, periodontal actives, abrasives, breath freshening agents, malodour control agents, tooth desensitizers, salivary stimulants, and combinations thereof. It is understood that while general attributes of each of the above categories of actives may differ, there may some common attributes and any given material may serve multiple purposes within two or more of such categories of actives.

Actives useful herein are present in the compositions of the present invention in safe and effective (clinically efficacious) amounts. A “safe and effective” and/or “clinically efficacious” amount of an active is an amount that is sufficient to have the desired therapeutic or prophylactic effect in the human or lower animal subject to whom the active is administered, without undue adverse side effects (such as toxicity, irritation, or allergic response), commensurate with a reasonable benefit/risk ratio when used in the manner of this invention. The specific safe and effective amount of the active will vary with such factors as the particular condition being treated, the physical condition of the subject, the nature of concurrent therapy (if any), the specific active used, the specific dosage form, the carrier employed, and the desired dosage regimen.

In various embodiments, the compositions of the present invention comprise an optional whitening agent. As further discussed below, a “whitening agent” is a material which is effective to effect whitening of a tooth surface to which it is applied. In various embodiments, the compositions optionally further comprise a non-peroxide whitening agent. Non-peroxide whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite.

The compositions optionally comprise additional orally acceptable antimicrobial agents. Such actives include 8-hydroxyquinoline and salts thereof; zinc and stannous ion sources such as zinc citrate, zinc sulphate, zinc glycinate, zinc gluconate, zinc lactate, sodium zinc citrate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride,); bisguanides, such as chlorhexidine digluconate, hexetidine, octenidine, and alexidine; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; grapeseed extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; and mixtures thereof. A further illustrative list of useful antibacterial agents is provided in U.S. Pat. No. 5,776,435, Gaffar, et al., issued Jul. 7, 1998.

The compositions of the present invention optionally comprise a tartar control (anticalculus) agent. Tartar control agents among those useful herein include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts include monobasic, dibasic and tribasic sodium phosphates, sodium tripolyphosphate (the preferred active), tetrapolyphosphate, mono-, di-, tri- and tetrasodium pyrophosphates, sodium trimetaphosphate, sodium hexametaphosphate and mixtures thereof, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J. One or more anticalculus agents are optionally present in an anticalculus effective total amount, typically about 0.001% to about 50%, for example about 0.05% to about 25% or about 0.1% to about 15%.

The compositions of the present invention optionally comprise a fluoride ion source useful, for example, as an anti-caries agent. Any compatible orally acceptable fluoride ion source can be used, including potassium fluoride, sodium fluoride (the preferred active), ammonium fluorides, monofluorophosphates, indium fluoride, stannous fluoride, and mixtures thereof. In various embodiments, water-soluble fluoride ion sources are used. In another embodiment, an amine fluoride, such as olaflur (N′-octadecyltrimethylendiamine-N,N,N′-tris(2-ethanol)-dihydrofluoride), provides fluoride ion to the composition. One or more fluoride ion sources are optionally present in an amount providing a total of about 0.001% to about 2%, for example about 0.005% to about 1% or about 0.01% to about 0.3%, of fluoride ions.

The compositions of the present invention optionally comprise an abrasive. In various embodiments, an abrasive is useful for example as a polishing agent. Any orally acceptable abrasive can be used, but type, fineness (particle size) and amount of abrasive should be selected so that tooth enamel is not excessively abraded in normal use of the composition. Suitable abrasives include silica, for example in the form of silica gel, hydrated silica or precipitated silica, alumina, insoluble phosphates, calcium carbonate, resinous abrasives such as urea-formaldehyde condensation products, and mixtures thereof. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. Average particle size of an abrasive, if present, is generally about 0.1 to about 30 microns, for example about 1 to about 20 microns or about 5 to about 15 microns.

The compositions of the present invention optionally comprise an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

The compositions of the present invention optionally comprise a saliva stimulating agent, useful for example in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in a saliva stimulating effective total amount.

The compositions of the present invention optionally comprise a breath freshening agent. Any orally acceptable breath freshening agent can be used, including, without limitation, zinc salts such as zinc gluconate, zinc citrate, zinc lactate, zinc chloride, or zinc chlorite, beta-ionone, and mixtures thereof. One or more breath freshening agents are optionally present in a breath freshening effective total amount.

The compositions of the present invention optionally comprise an anti-inflammatory agent. Any orally acceptable anti-inflammatory agent can be used, including steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone, and mixtures thereof.

The compositions of the present invention optionally comprise an H₂ antagonist. H₂ antagonists useful herein include cimetidine, etintidine, ranitidine, ICIA-5165, tiotidine, ORF-17578, lupititidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408.4, and mixtures thereof.

The compositions of the present invention optionally comprise a desensitizing agent. Desensitizing agents useful herein include potassium citrate, potassium chloride, potassium tartrate, potassium bicarbonate, potassium oxalate, potassium nitrate, strontium salts, and mixtures thereof. Alternatively (or, in addition) a local or systemic analgesic such as aspirin, codeine, acetaminophen, sodium salicylate or triethanolamine salicylate can be used.

The compositions of the present invention optionally comprise a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), fish oil (including components thereof such as omega-3 (N-3) polyunsaturated fatty acids, eicosapentaenoic acid and docosahexaenoic acid), coenzyme Q10, and mixtures thereof.

The compositions of the present invention optionally comprise proteins. Suitable proteins include milk proteins and enzymes such as peroxide-producing enzymes, amylase, plaque-disrupting agents such as papain, glucoamylase, glucose oxidase, and “next generation” enzymes.”

In a more specifically detailed formulated oral care composition embodiment,

the first phase comprises an aqueous blend of
from about 0.05 to about 0.8 weight percent sodium saccharin,
from about not greater than 0.243 weight percent sodium fluoride,
from about not greater than 0.8 weight percent xanthan gum,
from about 0.05 to about 1.5 weight percent sodium carboxymethyl cellulose gum,
from about 0.005 to about 1 weight percent carrageenan gum,
from about 0.005 to about 1 weight percent propylene glycol,
from about 8 to about 30 weight percent glycerin, from about 8 to about 30 weight percent sorbitol,
from about 0.5 to about 8 weight percent methyl vinyl ether/maleic anhydride copolymer,
sodium hydroxide sufficient for providing the measured pH of from about 7 to about 9,
from about 0.2 to about 1 weight percent 2,4,4′-trichloro-2′-hydroxydiphenyl ether,
from about not greater than 5 weight percent sodium lauryl sulfate,
from about 8 to about 40 weight percent abrasive silica,
from about 0.05 to about 5 weight percent thickening silica,
from about 0.05 to about 1.5 weight percent titanium dioxide,
from about 0.5 to about 2 weight percent flavorant, and
from about not greater than 0.1 weight percent butylated hydroxy toluene;

the second phase comprises an aqueous blend of from about 0.20 to about 0.25 weight percent sodium saccharin,
from about 0.243 to about 0.486 weight percent sodium fluoride,
from about 0.5 to about 4 weight percent hydrogen peroxide,
from about 0.01 to about 1 weight percent phosphoric acid,
from about 0.1 to about 0.8 weight percent xanthan gum,
from about 0.05 to about 3 weight percent acrylic acid gum,
from about 10 to about 50 weight percent glycerin, not greater than 4 weight percent methyl vinyl ether/maleic anhydride copolymer,
not greater than 0.5 weight percent 2,4,4′-trichloro-2′-hydroxydiphenyl ether,
not greater than 5 weight percent sodium lauryl sulfate,
from about 0.05 to about 2 weight percent abrasive silica,
not greater than 5 weight percent thickening silica,
not greater than 1.5 weight percent titanium dioxide,
from about 0.5 to about 2 weight percent flavorant, and
from about 0.005 to about 0.1 weight percent butylated hydroxy toluene;
   and the methyl vinyl ether/maleic anhydride copolymer has the structural formula,

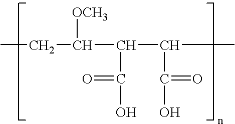

where n provides a molecular weight from about 100 to about 1,000,000.

Methods of Manufacture

The compositions of the present invention are made by any of a variety of methods, including adding and mixing the ingredients of the composition in a suitable vessel such as a stainless steel tank provided with a mixer. In one embodiment, the triclosan is admixed into a first orally acceptable aqueous vehicle to provide a first phase of a dentifrice; the hydrogen peroxide is admixed into a second orally acceptable aqueous vehicle to provide a second phase of the dentifrice; the first phase is stored in a first enclosure; the second phase is stored in a second enclosure; and the first phase is expelled from the first enclosure and the second phase is expelled from the second enclosure just prior to application to the teeth so that the first phase and the second phase are expelled to provide an amalgam where the amalgam has at least one first portion comprising the first phase, the amalgam has at least one second portion comprising the second phase, and each first portion is in fluid interface with at least one second portion. This embodiment is preferably provided to the consumer in the form of an oral care kit providing (a) a first chamber (the first storage enclosure for storing the first phase of the amalgam) having a first outlet in fluid communication with the first chamber for discharge of a first dentifrice (the first amalgam phase) from the first chamber and (b) a second chamber (the second storage enclosure for storing the second phase of the amalgam) having a second outlet in fluid communication with the second chamber for discharge of a second dentifrice (the second amalgam phase) from the second chamber. The second outlet is proximate to the first outlet so that, during simultaneous discharge of the first dentifrice from the first chamber through the first outlet and of the second dentifrice from the second chamber through the second outlet, discharged first dentifrice fluidly interfaces with discharged second dentifrice to form the amalgam. Such a system is also denoted herein as a dual compartment toothpaste tube. Preferably, the phases are visually distinguishable and equal amounts of each phase are delivered into the amalgam so that the consumer has a convenient basis for ascertaining that both phases are being appropriately delivered and that rapid intermixing of the phases will occur as the amalgam is brushed against the teeth.

Additional ingredients such as flavorant, coloring or sweeteners are added at any point during the mixing process but in various embodiments such ingredients are preferably added last or close to last.

Methods of Use

The present invention provides methods for cleaning and treating a tooth surface using compositions according to the present invention. As referred to herein, “tooth” or “teeth” refers to natural teeth, dentures, dental plates, fillings, caps, crowns, bridges, dental implants, and the like, and any other hard surfaced dental prosthesis either permanently or temporarily fixed within the oral cavity.

In various embodiments, compositions of the present invention are also used for the treatment or prevention of disorders in the oral cavity, including cavity prevention, whitening, plaque prevention or reduction, gingivitis prevention or reduction, tartar control, sensitivity prevention or reduction, breath malodor prevention or reduction, and stain prevention. Compositions of the present invention may also be used for the treatment or prevention of systemic disorders, such as the improvement of overall systemic health characterized by a reduction in risk of development of systemic diseases, such as cardiovascular disease, stroke, diabetes, severe respiratory infection, premature and low birth weight infants (including associated post-partum dysfunction in neurologic/developmental function), and associated increased risk of mortality. Such methods include those disclosed in U.S. Patent Publication 2003/0206874, Doyle et al., published Nov. 6, 2003.

The present invention is further illustrated through the following non-limiting example.

### EXAMPLE

Toothpaste formulations are prepared according to the formulations in weight percent given in Tables 1 and 2. Table 1 shows A, B, C, and D formulation detail; Table 2 shows detail in two control formulations.

TABLE 1

| | FORMULA | | | | | | | |
|---|---|---|---|---|---|---|---|---|
| | A | A | B | B | C | C | D | D |
| SIDE | 1 | 2 | 1 | 2 | 1 | 2 | 1 | 2 |
| FORM | paste | gel | paste | gel | paste | gel | paste | gel |
| Sodium | 0.3 | 0.25 | 0.3 | 0.25 | 0.3 | 0.25 | 0.3 | 0.25 |
| Saccharin | | | | | | | | |
| Sodium Fluoride | — | 0.486 | — | 0.486 | — | 0.486 | — | 0.486 |
| Hydrogen | — | 5.71 | — | 5.71 | — | 5.71 | — | 5.71 |
| Peroxide (35% | | | | | | | | |
| soln) | | | | | | | | |
| H3PO4 | — | 0.1 | — | 0.1 | — | 0.1 | — | 0.1 |
| (phosphoric | | | | | | | | |
| acid) | | | | | | | | |
| Xanthan (gum) | — | 0.4 | — | 0.4 | — | 0.4 | — | 0.4 |
| Carbopol 974 | — | 2.1 | — | 2.1 | — | 2.1 | — | 2.1 |
| gum (polymer of | | | | | | | | |
| acrylic acid) | | | | | | | | |
| Sodium | 0.5 | — | 0.5 | — | 0.5 | — | 0.5 | — |
| carboxymethyl | | | | | | | | |
| cellulose gum | | | | | | | | |
| Carrageenan | 0.2 | — | 0.2 | — | 0.2 | — | 0.2 | — |
| gum | | | | | | | | |
| Propylene | 0.5 | — | 0.5 | — | 0.5 | — | 0.5 | — |
| Glycol | | | | | | | | |
| Polyethylene | — | — | — | 10 | — | — | — | — |
| Glycol 600 | | | | | | | | |
| Glycerin | 14 | 40 | 14 | 40 | 14 | 40 | 14 | 40 |
| Sorbitol | 7 | — | 7 | — | 7 | — | 7 | — |
| Gantrez * | 30 | — | 30 | — | 30 | — | 30 | — |
| NaOH (50%) | 3.35 | — | 3.35 | — | 2.4 | — | 2.4 | — |
| Triclosan | 0.6 | — | 0.6 | — | 0.6 | — | 0.6 | — |
| SLS (Sodium | 3 | — | 3 | — | 3 | — | 3 | — |
| Lauryl Sulfate) | | | | | | | | |
| Thickening | 10 | 0.3 | 10 | 0.3 | 10 | 0.3 | 10 | 0.3 |
| silica 115 | | | | | | | | |
| Thickening | 2 | — | 2 | — | 2 | — | 2 | — |
| silica 165 | | | | | | | | |
| Abrasive silica | 22 | — | 22 | — | 22 | — | 22 | — |
| 650 | | | | | | | | |
| TiO2 (titanium | 0.5 | — | 0.5 | — | 0.5 | — | 0.5 | — |
| dioxide) | | | | | | | | |
| FD&C Blue #1 | — | 0.035 | — | 0.035 | — | 0.035 | — | 0.035 |
| Flavor | 1 | 1 | 1 | 1 | 1 | 1 | 1 | 1 |
| BHT (Butylated | — | 0.03 | — | 0.03 | — | 0.03 | — | 0.03 |
| Hydroxy | | | | | | | | |
| Toluene) | | | | | | | | |
| Water | balance | balance | balance | balance | balance | balance | balance | balance |
| PH (10% | 8.4 | 4.2 | 8.4 | 4.4 | 7.4 | 4.4 | 7.4 | 4.2 |
| solution) | | | | | | | | |

• methylvinyl ether-maleic anhydride copolymer 13 percent solution

TABLE 2

| | | CONTROL 2 |
|---|---|---|
| | CONTROL 1 | Single tube |
| | Single tube | formulation |
| FORMULA | formulation | (negative control) |
| Sodium Saccharin | 0.3 | 0.3 |
| Sodium Fluoride | 0.243 | 0.1 |
| Sodium | | 0.76 |
| Monofluorophosphate | | |
| Sodium | 1.1 | 0.9 |
| carboxymethyl | | |
| cellulose gum | | |
| carrageenan gum | 0.4 | |
| Propylene Glycol | 0.5 | |
| Polyethylene Glycol | | 2 |
| 600 | | |
| Glycerin | 20 | 10.101 |
| Sorbitol | 20.85 | 15 |
| Gantrez * | 15 | |
| NaOH (50% solution) | 1.2 | |
| Tetrasodium | | 0.25 |
| pyrophosphate | | |
| Triclosan | 0.3 | |
| SLS (Sodium Lauryl | 1.5 | 1.5 |
| Sulfate) | | |
| Poloxamer 407 | | 0.2 |
| Thickening silica 115 | 20 | |
| Thickening silica | 1.5 | |
| 165 | | |
| Dicalcium phosphate | | 48 |
| TiO2 (Titanium | 0.5 | |
| Dioxide) | | |
| Flavor | 1 | 0.95 |
| Water | balance | Balance |
| PH (10% solution) | 7.2 | 8 |

• methylvinyl ether-maleic anhydride copolymer 13 percent solution

Sodium carboxymethyl cellulose is provided as Sodium CMC2000S.
carrageenan gum is provided as Viscarin PS298.
Abrasive high cleaning silica 650 is provided as Sylodent XWA 650.
Abrasive silica 115 is provided as Zeodent 115 and thickening silica 165 is provided as Zeodent 165.

Note that the Control 1 formulation of Table 1 is formulated to provide an antibacterial active (in this study, triclosan) while being devoid of polyethylene glycol. In this regard, the Control 1 formulation is a commercially available triclosan-containing dentifrice devoid of polyethylene glycol. The Control 2 formulation of Table 1 contains polyethylene glycol and is formulated to essentially provide “placebo” type toothpaste devoid of an antibacterial active (in this study, triclosan).

Efficacy in Table 1 formulations is measured according to the experimental plans of the following Examples.

### MIC Value Test

An MIC value test is conducted (Minimum Inhibitory Concentration: measure of antibacterial activity; the lower the Score, the better the antibacterial activity—MIC determines the lowest concentration for which there is complete inhibition of bacterial growth) on the streptococcus mutans bacteria organism. Results indicate the following minima for toothpaste concentrations efficacious against inhibiting growth of streptococcus mutan.

Formula A 0.23 ppm

Formula B 0.46 ppm

Formula C 0.93 ppm

Formula D 0.46 ppm

Control 1 0.93 ppm

Note that Control 1 and Formula C both have polyethylene glycol and do not show the same efficacy in the MIC test as Formulas A, B, and D (essentially devoid of polyethylene glycol).

### Artificial Mouth

In vitro model for dental plaque formation. This model uses a chemostat, which provides continuous sources of multiple strains of the following oral bacteria, Actinomyces naeslundii, Streptococcus mutans, Fusobacteria nucleatum. Veillonella parvula, Porhyromonas gingivalis, Lactobacillus.casei, and Streptococcus sanguis.

The mixture is pumped through an 8 chamber flow cell, containing 8 hydroxyapatite disks each, in order for plaque to form on the disks. Each chamber in the flow cell is treated with 10 mls of a dentifrice supernatant for a total of four times during a three day period of time. The dentifrice supernatant set is obtained by making a set of dentifrice slurries of, independently, 10 grs of paste from each of Formulas A, B, C, Control 1, and Control 2 in 20 grs of water for each slurry; each treatment is applied by centrifuging for 10 minutes at 20,000 rpm. The formation of plaque is measured by removing the plaque from off of the disks by treating each disk with 0.25% trypsin for 45 minutes in a 37° C. water bath. The disks are gently vortexed to remove bacteria, and the disks are discarded. The bacterial mixture is sonicated for dispersion and the optical density is measured at 610 nm in a spectrophotometer. The optical density values for mixtures treated with Formulas A, B, and C are then compared against optical density values for mixtures treated with Control Formulas (Formulas Control 1 and Control 2). Results indicate the following percentage reduction in optical density for the mixtures:

Formula A 78.13

Formula B 28.02

Formula C 47.03

Control 1 35.16

Control 2 0

Note that Control 2, Formula B, and Formula C formulations contain polyethylene glycol and do not show the same efficacy in the MIC test as Formula A (essentially devoid of polyethylene glycol).

Overall, MIC and Artificial Mouth studies (anti-efficacy studies) show that Formula A without polyethylene glycol performs significantly better that formulas with polyethylene glycol.

### Whitening Studies

Human molar teeth free of defects are selected and sent out for artificial staining and embedding in an acrylic impression material. A statistical set of 5 teeth is used for each dentifrice tested. Before each experiment, the artificially stained teeth are cleaned and pumiced to remove surface (extrinsic stains). An initial color reading (CIELAB L*a*b values) are taken for each tooth and are then divided into sets containing 5 statistically balanced teeth based on their initial L* values.

Brushing Model

Prophied, artificially stained human teeth are embedded in an impression clay material (Kerr Impression Compound, Romulus, Ml) into prefabricated brushing trays. Teeth are kept fully immersed in water at all times except during brushing. The toothpaste slurries are prepared by 1:1 dilution with water. Slurries for the dual chamber dentifrices (Colgate Simply White) are prepared as follows: the gels in the two different chambers are diluted with an equal amount of water and kept separately. The diluted gels are then mixed in equal amounts right before every brushing set. The slurry thus formed is homogenized using a homogenizer for the active ingredients to be fully mixed. The same procedure was used to prepare the slurries for Colgate Total Plus Whitening with hydrogen peroxide where the hydrogen peroxide component (blue gel) is diluted with water in 1:1 portions. The triclosan components (white gels A and B) are also diluted with water in 1:1 portions. The diluted hydrogen peroxide component is then mixed with the diluted triclosan component: (gel A) to form TPW+Gel A and with the triclosan component (gel B) to form TPW+Gel B. Dentifrice slurry (20 g) is used for each set of brushing. The teeth are mechanically brushed for a total of four sets of seven brushing cycles (300 strokes in 3 minutes per cycle). Brushing is performed on an in-house apparatus using Colgate toothbrushes under a weight pressure of 116.88 g. At the end of the brushing period, teeth are washed thoroughly with water and readings are taken using the “Spectroshade” Spectrophotometer.

Color Measurement

The color of the teeth is measured using a Spectroshade Spectophotometer (Niederhasli. Switzerland). Measurements are performed by aligning the aperature of the camera with the exposed part of the tooth specimen. The color of teeth is recorded in L*a*b color factors where L* represents the change in lightness shade, a* is a measure of blueness and b* is a measure of yellowness. The L*a*b* values are measured initially, and also after completing the treatments for the brushing and the soaking studies. To calculate the degree of whitening at any time point, the final measurements for each tooth (L*a*b) is evaluated against its initial color values (L*a*b)ₜ using equation (1). The higher the composite color change, the greater the whitening effect.
Change in E=[(Lₜ−Lᵢ)²+(aₜ−aᵢ)²+(bₜ−bᵢ)²]^{1/2} Equation 1

Statistical analyses are done using ANOVA, Fishers pair-wise comparisons are at the 95% confidence level.

Table 3 shows initial values and values after 28 treatments for Formulas Control 1 (control) and B, and C.

TABLE 3

| | Initial | Initial | Initial | Final | Final | Final |
|---|---|---|---|---|---|---|
| Sample | Color | Color | Color | Color | Color | Color |
| Equation 1 | Lᵢ | aᵢ | bᵢ | Lₜ | aₜ | bt |
| Variable | | | | | | |
| value | | | | | | |
| Control 1 | 56.05 | 1.25 | 12.2 | 58.59 | 1.31 | 14.56 |
| Formula B | 56.10 | 0.93 | 11.64 | 62.03 | 0.53 | 13.19 |
| Formula C | 56.93 | 0.85 | 12.75 | 63.15 | 0.45 | 14.53 |

Changes in Table 3 values are summarized in Table 4

TABLE 4

| Formulation | Change in L | Change in a | Change in b | Change in E |
|---|---|---|---|---|
| Control 1 | 2.54 +/− 0.84 | 0.06 +/− 0.2 | 2.36 +/− 0.98 | 3.56 +/− 0.92 |
| Formula B | 5.93 +/− 1.47 | −0.40 +/− 0.29 | 1.55 +/− 0.21 | 6.15 +/− 1.42 |
| Formula C | 6.22 +/− 1.56 | −0.39 +/− 0.42 | 1.79 +/− 0.60 | 6.51 +/− 1.60 |

Final changes in E (rightmost column in Table 4) indicate that Formulas B and C containing peroxide (efficacious against intrinsic stains) whiten teeth much better than the Control (Control 1) formulation that only contains the high silica abrasive (efficacious against extrinsic stains). The pH of the formulation does not appear to be directly significant in stain removal efficacy.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. An oral care composition, comprising:
(a) a first phase comprising a halogenated diphenyl ether, an antibacterial-enhancing agent, and a first orally acceptable aqueous vehicle; and
(b) a second phase comprising a peroxide compound, an antioxidant, and a second orally acceptable aqueous vehicle;
(c) wherein said first phase has a measured pH of from about 7 to about 9, and said second phase has a measured pH of from about 3.5 to about 6.5.

2. An oral care composition according to claim 1, wherein said halogenated diphenyl ether is 2,4,4′-trichloro-2′-hydroxydiphenyl ether.

3. An oral care composition according to claim 1, wherein said antibacterial enhancing agent is a methylvinyl ether-maleic anhydride copolymer having a molecular weight from about 100 to about 1,000,000.

4. An oral care composition according to claim 1, wherein said peroxide compound is hydrogen peroxide.

5. The composition of claim 1 wherein said antioxidant is butylated hydroxy toluene.

6. An oral care composition according to claim 1 further comprising sodium fluoride in at least one of said first phase and said second phase.

7. An oral care composition according to claim 1 further comprising silica in said second phase.

8. An oral care composition according to claim 1 further comprising silica in said first phase.

9. An oral care composition according to claim 1 further comprising sodium lauryl sulfate in said first phase.

10. An oral care composition according to claim 1 further comprising sodium lauryl sulfate in said second phase.

11. An oral care kit, comprising:
(a) a first chamber having a first outlet in fluid communication with said first chamber for discharge of a first dentifrice from said first chamber; and
(b) a second chamber having a second outlet in fluid communication with said second chamber for discharge of a second dentifrice from said second chamber;
(c) wherein said first dentifrice comprises 2,4,4′-trichloro-2′-hydroxydiphenyl ether and a methylvinyl ether-maleic anhydride copolymer admixed in a first orally acceptable aqueous vehicle, said second dentifrice comprises hydrogen peroxide and an antioxidant admixed in a second orally acceptable aqueous vehicle, said first dentifrice has a measured pH of from about 7 to about 9, and said second dentifrice has a measured pH of from about 3.5 to about 6.5, and said second outlet is proximate to said first outlet so that, during simultaneous discharge of said first dentifrice from said first chamber through said first outlet and of said second dentifrice from said second chamber through said second outlet, discharged first dentifrice fluidly interfaces with discharged second dentifrice to yield an amalgam such that said discharged first dentifrice provides at least one first portion of said amalgam, said discharged second dentifrice provides at least one second portion of said amalgam, and at least one said first portion is in fluid interface with at least one said second portion.

12. An oral care kit according to claim 11 wherein said methylvinyl ether-maleic anhydride copolymer antibacterial-enhancing agent has a molecular weight of about 100 to about 1,000,000.

13. An oral care kit according to claim 11 wherein said antioxidant is butylated hydroxy toluene.

14. An oral care kit according to claim 11 wherein at least one of said first dentifrice and said second dentifrice further comprise sodium fluoride.

15. An oral care kit according to claim 11 wherein said second dentifrice further comprises silica.

16. An oral care kit according to claim 11 wherein said first dentifrice further comprises silica.
